(19) United States
(12) Patent Application Publication
(10) Pub. No.: US 2008/0103560 A1
(43) Pub. Date: May 1, 2008

(54) ULTRAVIOLET INDICATOR LIGHT THERAPY DEVICE

(75) Inventors: Steven D. Powell, Orem, UT (US); Andrew Michael Smith, Orem, UT (US)

Correspondence Address: STOEL RIVES LLP - SLC, 201 SOUTH MAIN STREET, ONE UTAH CENTER, SALT LAKE CITY, UT, 84111, US

(73) Assignee: LUMIPORT, LLC, Provo, UT (US)

(21) Appl. No.: 11/744,355

(22) Filed: May 4, 2007

Related U.S. Application Data
(60) Provisional application No. 60/863,085, filed on Oct. 26, 2006.

(51) Int. Cl. A61N 5/06

(52) U.S. Cl. 607/88; 607/89; 607/94

(57) ABSTRACT

A phototherapy device includes a first light source configured to emit a wavelength of light within the visible to infrared portion of the electromagnetic spectrum and a second light source configured to emit a wavelength of light within the ultraviolet portion of the electromagnetic spectrum, the wavelength of light of the first light source being selected to treat a skin condition.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of one embodiment of a phototherapy device used in the treatment of skin conditions.

FIG. 2 is a plan view of a control panel of one embodiment of a phototherapy device.

FIG. 3 is a side elevation view of a recharging base station and a phototherapy device.

FIG. 4A is a front plan view of another embodiment of a phototherapy device.

FIG. 4B is a side elevation view of the phototherapy device of FIG. 4A.

FIG. 4C is a plan view of the back side of the phototherapy device of FIG. 4A.

FIG. 5 is a block diagram of a system for treating various skin conditions with a phototherapy device.

### DETAILED DESCRIPTION

It will be readily understood that the components of the embodiments as generally described and illustrated in the Figures herein could be arranged and designed in a wide variety of different configurations. In some cases, well-known structures, materials, or operations are not shown or described in detail. The following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

For this application, the phrases “connected to” and “coupled to” refer to any form of interaction between two or more entities, including mechanical, electrical, magnetic, electromagnetic, fluid, and thermal interaction. Two components may be coupled to each other even though they are not in direct contact with each other.

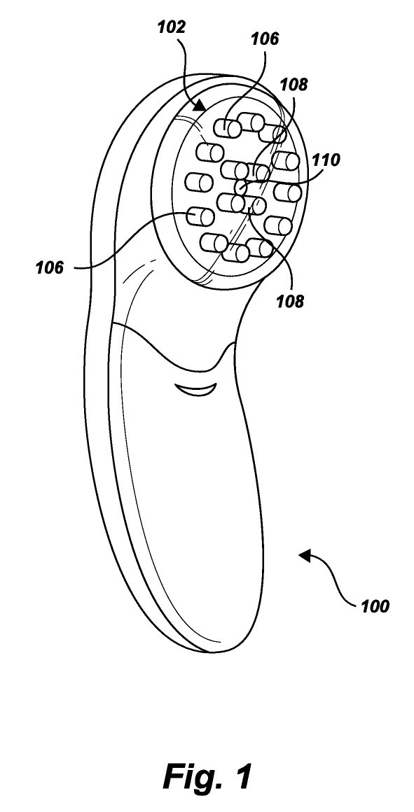

FIG. 1 represents one embodiment of a phototherapy device 100 used in the treatment of various skin conditions, as shown from a perspective view. Device 100 may comprise an array of light emitting diodes (“LEDs”) 102 disposed at one end of device 100 to provide light therapy treatment to a user in need thereof. Alternatively, different light sources may be used such as, but not limited to, lasers, incandescent lamps, fluorescent lamps, plasma arc lamps, and the like. The phototherapy device 100 may include a housing that can be easily gripped by a user.

The LED array 102 may comprise a first array of LEDs 106. For example, the LEDs arranged around the perimeter of the head of the device 100 may comprise the first array of LEDs 106. The LED array 102 may further comprise a second array of LEDs 108 disposed on the same surface as the first array, but, for example, disposed in the center portion of LED array 102. According to one embodiment, the first array 106 may comprise a number of LEDs that emit a wavelength of light selected to treat a skin condition. The wavelength of light may be disposed in the visible portion of the electromagnetic spectrum and/or the infrared portion of the electromagnetic spectrum.

The second array 108 may comprise a number of LEDs that emit light in the ultraviolet portion of the electromagnetic spectrum. UV LEDs are known in the art, such as the FoxUV™ LED, manufactured by the Fox Group of Deer Park, N.Y., which may emit ultraviolet light the range of 360-365 nm. Orange/red fluorescence (in the range of 580-680 nm) indicates the presence of P. acnes bacteria, one of the causes of acne.

The configuration of the first 106 and second 108 arrays of LEDs may differ in alternative embodiments. For example, the first array 106 may be disposed on a different surface than the second array 108. Alternatively, the LEDs of the first array 106 may be interspersed with the LEDs of the second array 108 as would be appreciated by those having skill in the art with the aid of the present disclosure.

Once the phototherapy device 100 is activated, the first array of LEDs 102 emit light in a narrow range of wavelengths. LEDs may be a desirable light source since they typically use less power, produce less heat, and have a longer life span than most incandescent lamps. Furthermore, LEDs are often an inexpensive alternative to wavelength selection compared to lamp and filter systems. However, in alternative embodiments, the light source may comprise a laser, lamp and filter arrangement, or other light sources and related assemblies for delivering light at a wavelength selected to treat a skin condition.

In one embodiment, the first array of LEDs 106 may all emit the same narrow range of wavelengths and the light emitted may be considered monochromatic. In alternative embodiments, multiple LED types may be used to emit various ranges of wavelengths. In yet other embodiments, multi-color LEDs may be used to emit more than one discrete range of wavelengths. For example, the multi-color LEDs may comprise bi-color, or bi-polar LEDs producing two discrete ranges of wavelengths. In other embodiments, the multi-color LEDs are tri-color LEDs producing three discrete ranges of wavelengths. As would be apparent to those having skill in the art, multi-color LEDs may be used which can produce more than three discrete wavelengths as the advancement of technology permits.

By way of example, in one embodiment, the first LED array 106 may produce a narrow band of wavelengths in the red portion of the visible electromagnetic spectrum as well as a narrow band of wavelengths in the blue portion of the visible electromagnetic spectrum, using either an array of monochromatic LEDs or multi-color LEDs, or both. The red wavelengths may range between about 630 nanometers and about 680 nanometers, while the blue wavelengths may range between about 400 nanometers and about 470 nanometers. In one embodiment, the red band is between about 650 to about 670 nanometers and the blue band is between about 405 to about 420 nanometers. Moreover, the LED array 108 may be capable of producing just red wavelengths at one time, or just blue wavelengths, or both red and blue wavelengths simultaneously.

The light therapy device may be used to treat a variety of skin conditions. The first LED array 106 of device 100 may be directed toward or placed on a region of skin having a particular skin condition so that the skin may be treated with light therapy. The first LED array 106 may produce specific wavelengths to treat a number of skin conditions. For example, for the treatment of acne both blue wavelengths (about 400 to about 470 nanometers) and red wavelengths (about 630 to about 680 nanometers) may be used. Furthermore, for the treatment of acne, the first LED array 106 may provide twice as much exposure to blue wavelengths than to red wavelengths in a single treatment event. Relative exposures of red and blue wavelengths may be determined through a quantifiable value such as light intensity or duration of exposure.

In order to treat wrinkles in the skin, blue, red and yellow wavelength bands may be used. The blue and red wavelengths may range between about 400 to about 470 nanometers and about 630 to about 680 nanometers, respectively. The yellow band of wavelengths may be between about 530 nanometers and about 600 nanometers.

In treating rosacea, a yellow range of wavelengths may be used between about 530 and about 600 nanometers.

In treating sun spots, a yellow range of wavelengths (about 530 to about 600 nanometers) may be used. For alternative forms of sun damage, a red band (about 630 to about 680 nanometers) may be employed.

Blue light (between about 400 and about 470 nanometers) may be used to treat and kill bacteria that may cause various forms of skin blemishes, such as acne.

Inflammation may be treated by exposing affected skin to red wavelengths (about 630 to about 680 nanometers) and also to infrared wavelengths, which may range from about 800 nanometers to about 1000 nanometers.

Lesions in the skin may be treated by illuminating the affected area with red wavelengths (about 630 to about 680 nanometers) and infrared wavelengths (about 800 to about 1000 nanometers).

Skin blemishes may be treated through exposure to red, blue and yellow wavelengths. As discussed above, the wavelength ranges may be about 630 to about 680 nanometers for red, about 400 to about 470 nanometers for blue, and about 530 to about 600 nanometers for yellow.

LEDs that emit a band of wavelengths in the green portion of the visible electromagnetic spectrum may also be used in treating sun spots, rosacea and wrinkles. The wavelength range associated with green light may range between about 500 nanometers and about 530 nanometers. LED light therapy may also be used in treating dead skin and other skin problems.

The second LED array 108 may comprise a series of LEDs that emit a narrow band of wavelengths in the ultraviolet portion of the electromagnetic spectrum. Ultraviolet light may cause porphyrins produced by bacteria present on the skin to fluoresce orange-red (580-680 nm). The second LED array 108 consequently acts as an ultraviolet indicator to indicate the presence and location of bacteria for light therapy treatment.

The fluorescence emitted by bacteria-produced porphyrins may be visually detectable by a user to identify the presence and location of bacteria on a user's skin. However, in some embodiments the phototherapy device 100 includes a detector 110 capable of detecting the presence and location of the bacteria. For example, an exemplary detector 110 may be configured to monitor a wavelength of light that is emitted when bacteria-produced porphyrins fluoresce. When that wavelength of light is detected from fluorescing porphyrins, the user may be alerted by a signal, such as a visual display, light indicator or audible signal. Various detectors 110 may be used as would be appreciated by those having skill in the art, such as CCD arrays, photomultiplier tubes, and other fluorescence detectors, which may include various filters for isolating a desired range of wavelengths. One particular example of a detector 110 is disclosed in U.S. Pat. No. 5,760,407 to Michael Paul Aronson, which is incorporated herein by reference in its entirety.

In one embodiment, the second LED array 108 may be illuminated to determine the presence and location of bacteria on a user's skin before light therapy treatment, after light therapy treatment, or between light therapy treatment intervals. After the bacteria location has been identified, the first LED array 106 may be illuminated to treat the area of skin having detectable bacteria levels.

Alternatively, the first and second LED arrays 106, 108 may be used simultaneously. For example, the second LED array 108 may include one or more LEDs that produce ultraviolet light in the 300-350 nm range, which may be efficacious in killing acne on the surface of the skin. Of course, with shorter wavelengths of ultraviolet radiation, care should be taken to prevent overexposure. For example, the second LED array 108 may be configured with a separate timer to shut off earlier than the first LED array 106.

In yet other embodiments where multi-color LEDs are used, a single LED may emit light in both a visible portion of the electromagnetic spectrum and in an ultraviolet portion of the electromagnetic spectrum. The control system of the light therapy device 100 may cause only ultraviolet light to be emitted when determining the presence and location of bacteria, while visible or infrared light is emitted to treat any affected areas of the skin.

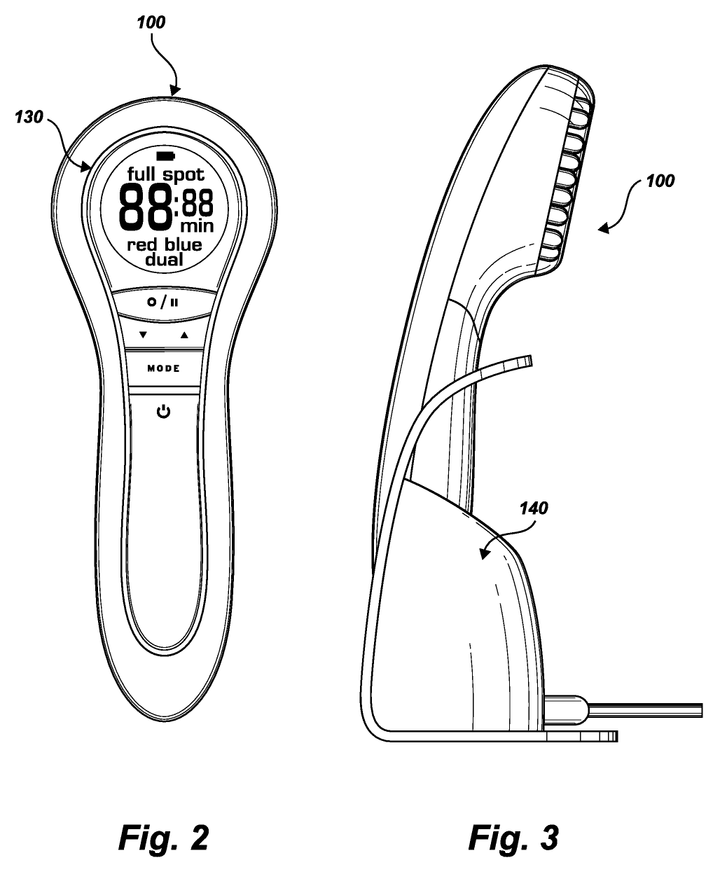

Referring to FIG. 2, a control panel 130 is shown on the backside of the phototherapy device 100. The control panel 130 may control the duration of light therapy treatment. The control panel 130 may also set the conditions of light treatment. For example, control panel 130 may select which wavelengths are used in a particular treatment, i.e., blue, red, yellow, green, infrared, and combinations thereof as discussed herein. Furthermore, in some embodiments, the control panel 130 may be programmed to allow device 100 to emit a combination of wavelengths simultaneously to treat different skin conditions at the same time. Control panel 130 may also control the intensity of the light emitted from the first LED array. The intensities of each color may also be varied independently in some embodiments. The control panel 130 may also control the functionality of the ultraviolet light source. The ultraviolet light source may be controlled with a simple on/off setting or in a manner similar to the first LED array.

Programming for the control panel 130 may be built-in and/or user-configurable. For example, the control panel 130 may include built-in parameters for wavelength, time, and intensity for treating a number of skin conditions. The user may be allowed to modify those parameters for his or her unique skin types and/or conditions. In certain embodiments, the control panel 130 may include a communications port, such as a universal serial bus (USB) port, for interfacing the device 100 to a computer (not shown). Using software on the computer, the user may be able to modify any of the built-in parameters or even download parameters (including complete treatment regimens for different skin conditions) from a website. In one implementation, the website may allow customized treatment regimens to be created and stored for different users, which could be downloaded to the device 100 as needed.

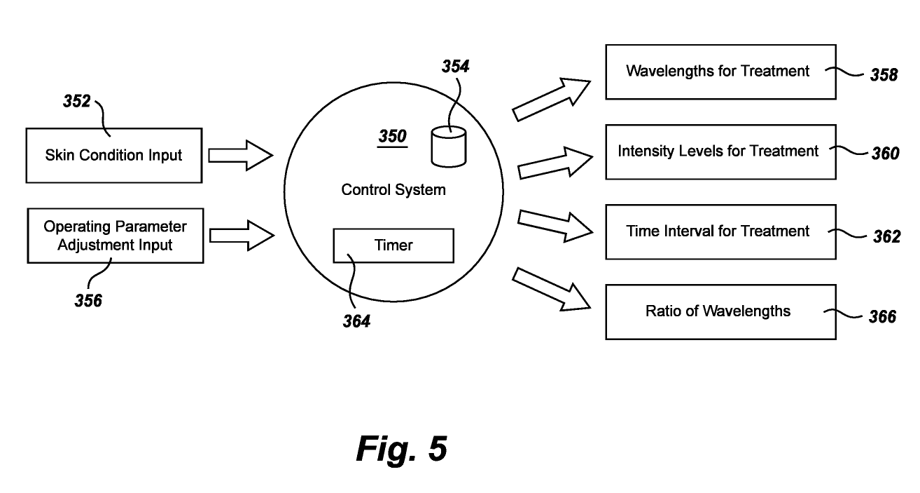

Other features of the control panel 130 may include a power button, LED treatment on/off button, UV indicator on/off button, an interrupt button, a battery power display, timer, wavelength selection and/or other alternative displays. For example, a LCD screen may optionally prompt a user for input or indicate operating status, etc. One embodiment of a control system associated with the control panel 130 of device 100 is described in greater detail in conjunction with FIG. 5.

In one configuration, the control system is in electrical communication with the detector 110, such that one or more of the parameters may be affected by input from the detector 110. For example, if the detector 110 discovers a relatively higher amount of fluorescence (indicating a greater than normal concentration of P. acnes bacteria), the duration (time) and/or intensity parameters may be correspondingly increased. On the other hand, a lower amount of detected fluorescence may result in a reduction of the duration and/or intensity parameters.

In one embodiment, the LEDs are illuminated only when the device 100 is in close proximity to or in contact with the user's skin. In one embodiment, this may prevent the user from inadvertently damaging his or her eyes by looking directly into the second LED array 108. For example, a pressure sensor may be placed adjacent the LED array in order to sense when the device 100 is pressed against the user's skin. In other embodiments, touch sensors, such as TouchCells™, manufactured by TouchSensor Technologies, LLC, may be used. When voltage is applied to the touch sensor, an electric field is created. The field emanates through any dielectric substrate such as glass or plastic. When a conductive mass enters the field, the sensor detects the change and indicates an event has occurred. The input stimulus to the field can take the form of contact with the user's skin. Various other proximity sensors and/or photoelectric sensors, as known in the art, could also be used.

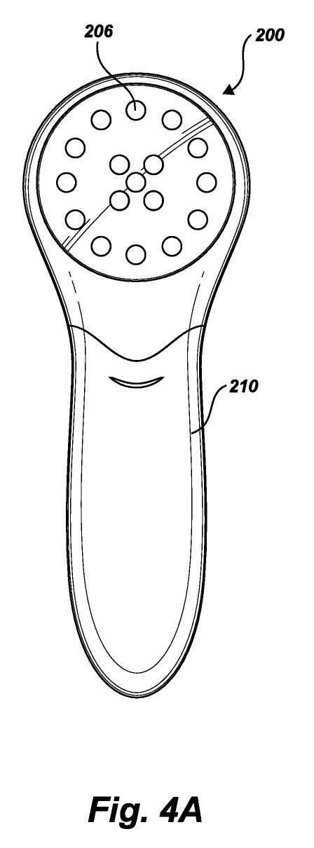

FIG. 3 depicts the phototherapy device 100 from a side elevation view within a docking station 140. Docking station 140 may comprise a recharging base station. Device 100 may be powered by an internal portable power source, such as a battery. The battery power source may provide device 100 with sufficient power that AC power is not required.

When the phototherapy device 100 is cradled within docking station 140 as depicted, the docking station 140 may have contact points that are in electronic communication with contact points of the phototherapy device 100. The docking station 140 is also connected to an AC power supply through a power cord. Alternatively, the phototherapy device 100 may be recharged using an AC adapter.

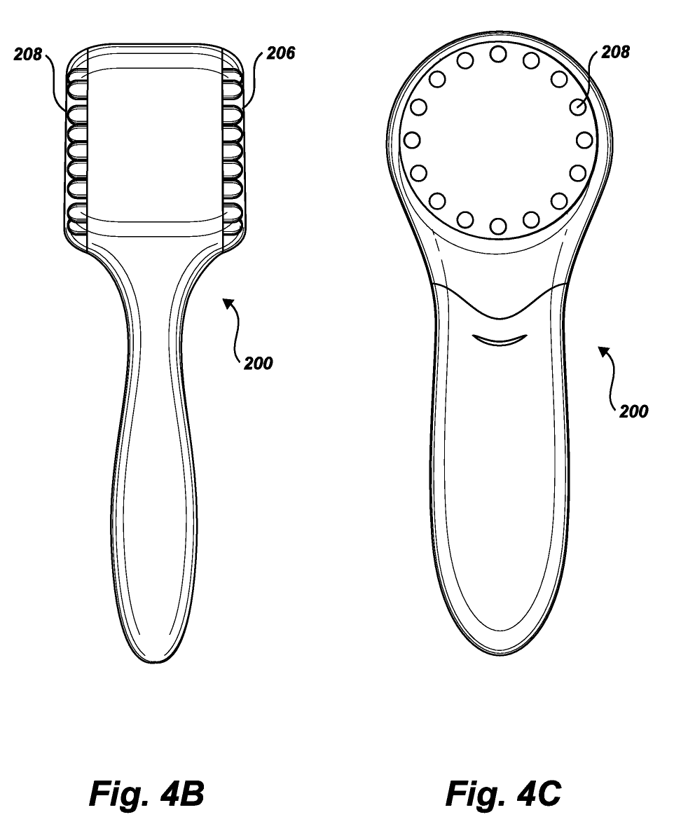

FIG. 4A represents another embodiment of a phototherapy device 200, as shown from a front plan view. The housing 210 of the phototherapy device 200 may be ergonomically shaped so a user can easily grip it. The device 200 may include a first LED array 206 disposed at one end of device 200 to provide light therapy treatment to a user in need thereof. The first LED array 206 may work similarly as the first LED array 106 described in conjunction with the description accompanying FIG. 1. In some embodiments, the first LED array 206 may have a cover or lens which is transparent to visible light, but functions to filter or diffuse ultraviolet light that may inadvertently be emitted from the first LED array 206.

FIG. 4B represents the phototherapy device 200 of FIG. 4A, from a side elevation view. The first LED array 206 is disposed on a first surface of device 200. A second LED array 208 is disposed on a second surface of device 200. The second LED array 208 may comprise a series of LEDs emitting ultraviolet light. In alternative embodiments a light source other than LEDs may be used to emit ultraviolet light, such as a lamp, laser and the like.

In the embodiment depicted, the second surface having the second LED array 208 is disposed on an opposite side from the first surface. One having skill in the art would appreciate that the second LED array 208 may be disposed at other locations on the device 200, such at its top end, or on the same surface as the first LED array 206.

FIG. 4C represents a rear plan view of the phototherapy device 200, displaying the second LED array 208 having ultraviolet-emitting LEDs. As discussed previously, the second LED array 208 may be used on an area of a user's skin that have been or will be treated with the first LED array 206. Ultraviolet light may cause bacteria-produced porphyrins present on the skin to fluoresce. The second LED array 208 consequently acts as an ultraviolet indicator to indicate the presence and location of bacteria for light therapy treatment.

In one embodiment, a user may shine the second LED array 208 onto an area of skin to determine if and where bacteria exists. If sufficient bacteria exist to cause detectable fluorescence, the user may then rotate the phototherapy device 200 and shine the first LED array 206 onto the area of skin with detectable bacteria.

FIG. 5 is a block diagram of a control system 350 for treating various skin conditions with a phototherapy device, such as devices 100, 200. The control system 350 may be incorporated, in part, into a control panel as heretofore described. The control system 350 may receive various forms of user input in order to control various treatment modes of the phototherapy device.

For example, a user may provide input 352 indicative of a skin condition that a user desires to be treated by the device. Examples of various skin condition inputs 352 may include acne, rosacea, wrinkles, inflammation, sun spots or sun damage, bacteria, blemishes or lesions. A user may select one or more of a list of skin conditions to be treated and the control system 350 accesses operating parameters stored on a memory device 354 or database in machine readable form. The operating parameters of the phototherapy device that correspond with a particular light therapy treatment may be inputted by a manufacturer or programmer of the device, or alternatively a user may provide adjustment operating parameter input 356 in accordance with a customized LED skin treatment program.

The control system 350 accesses the memory device 354 containing multiple operating parameters and selects those corresponding to the skin condition input 352 received. The phototherapy device then runs according to the operating parameters corresponding with the selected skin condition input 352. One example of an operating parameter output of the control system 350 is a control signal corresponding to the specific wavelengths for treatment 358 of the skin condition selected. Accordingly, if acne is selected by the user, the control system 350 accesses the corresponding operating parameter that indicates both red and blue wavelengths are to be used for treatment. However, if the user selected rosacea as the skin condition to be treated, the wavelengths for treatment 358 may be in the yellow band (about 530 to about 600 nanometers).

Another form of output of the control system 350 is the operating parameter that indicates the intensity levels 360 for treatment of the skin condition selected. For example, in one embodiment the intensity levels of a LED may be 105 mW/cm². However, an exemplary alternative intensity level output 360 of 92 mW/cm² may be provided by the control system 350. A user may adjust the intensity level output 360 corresponding to a particular skin treatment for the first LED array. The user adjusts that particular operating parameter through input 356 indicating an increase or a decrease in intensity to treat more severe or less severe skin conditions, respectively. Additionally, a user may adjust the intensity level output 360 for the ultraviolet output of the second LED array. Intensity adjustments may be made, for example, in percentage increments such as ±5%, ±10%, ±15%, etc.

Another operating parameter that may be controlled is the time interval for treatment 362. An exemplary treatment session may last 15 minutes for some skin conditions. However, treatment session times may be less, such as between 3 and 15 minutes, depending upon the user input. The time interval for treatment 362 may be controlled by a timer 364, which may be embodied, for example, as a Real Time Clock (RTC). Once the skin condition input 352 is received and the corresponding operating parameters accessed, the indicated time interval 362 is controlled by the timer 364. Once the timer 364 reaches the time interval 362 indicated it automatically shuts off LED emission.

Additionally, the operating parameters corresponding to a skin condition input 352 may include wavelength ratio data 366. For example, when acne is selected as the skin condition to be treated, the operating parameters corresponding with the treatment of acne would indicate that twice as much exposure to blue wavelengths as compared to red wavelengths is desired. Consequently, the wavelength ratio 366 for acne would be 2:1, blue to red. The relative exposures of red and blue wavelengths may be determined through a quantifiable value such as light intensity or duration of exposure. Therefore, blue LED light may be emitted at twice the intensity of red LED light. Alternatively, the exposure time of blue LED light during a particular treatment interval would be twice as long as red LED light. This may be accomplished by pulsating blue LEDs twice as much as red LEDs, or by activating twice as many blue LEDs than red LEDs, or other methods known to those having skill in the art.

Accordingly, a user is able to control the wavelengths emitted, the intensity levels, the time intervals for treatment, and the relative ratio of wavelengths produced by simply selecting a particular skin condition. By selecting the skin condition, the control system 350 causes the LED phototherapy device to provide the appropriate colors, intensity, etc., for that skin condition.

The control system may be in electronic communication with a control panel, such as that discussed in conjunction with the description of FIG. 2. By way of example, the control panel may include a LCD display which may show an indication of the skin condition selected by the user and the associated operating parameters. In some embodiments, the display may show a countdown of time left or time elapsed for the particular light therapy treatment. Furthermore, an audible alert, such as a beep, may let the user know when the treatment event has ended.

While specific embodiments and applications of phototherapy personal care devices have been illustrated and described, it is to be understood that the disclosure is not limited to the precise configuration and components provided. Various modifications, changes, and variations apparent to those of skill in the art may be made in the arrangement, operation, and details of the devices and systems disclosed.

1. A phototherapy device, comprising:
a first light source configured to emit a wavelength of light within the visible to infrared portion of the electromagnetic spectrum; and
a second light source configured to emit a wavelength of light within the ultraviolet portion of the electromagnetic spectrum;
wherein the wavelength of light of the first light source is selected to treat a skin condition.

2. The phototherapy device of claim 1, wherein the first light source comprises at least one light emitting diode (LED).

3. The phototherapy device of claim 2, wherein the second light source comprises at least one LED configured to emit a wavelength of light in the ultraviolet portion of the electromagnetic spectrum which causes a byproduct of bacteria on the skin to fluoresce.

4. The phototherapy device of claim 3, further comprising a housing, such that the first light source comprises an array of LEDs disposed on a first surface of the housing and the second light source is disposed on a second surface of the housing.

5. The phototherapy device of claim 3, further comprising a housing, such that the first light source comprises an array of LEDs disposed on a first surface of the housing and the second light source is also disposed on the first surface of the housing.

6. The phototherapy device of claim 3, wherein the first light source and the second light source comprises at least one multi-color LED capable of emitting a first wavelength in the visible to infrared portion of the electromagnetic spectrum and a second wavelength in the ultraviolet portion of the electromagnetic spectrum.

7. The phototherapy device of claim 2, wherein the at least one LED of the first light source comprises a multi-color LED capable of emitting more than one discrete wavelength.

8. The phototherapy device of claim 2, wherein the first light source comprises an array of LEDs configured to emit a wavelength in a blue portion of the visible electromagnetic spectrum and a wavelength in a red portion of the visible electromagnetic spectrum.

9. The phototherapy device of claim 8, wherein the blue wavelength is between 400 nanometers and 470 nanometers and the red wavelength is between 630 nanometers and 680 nanometers.

10. The phototherapy device of claim 2, further comprising a control system to control the at least one LED according to operating parameters, the operating parameters including at least one of: intensity level of LED emission, duration of LED emission, or wavelength selection.

11. The phototherapy device of claim 10, wherein the control system controls the at least one LED in accordance with the operating parameters corresponding to treatment of a skin condition selected by the user.

12. The phototherapy device of claim 11, wherein the operating parameters include at least one time interval representing a length of time the LED illumination source emits light for treatment of each skin condition, and wherein the control system comprises a timer which is set according to the at least one time interval of the operating parameters corresponding to the selected skin condition, such that emission of the LED illumination source is automatically discontinued when the at least one time interval has elapsed.

13. The phototherapy device of claim 10, wherein the operating parameters further include at least one wavelength ratio representing how much of a quantifiable value of one wavelength is emitted relative to the quantifiable value of another wavelength.

14. The phototherapy device of claim 10, wherein the operating parameters are adjustable by a user.

15. The phototherapy device of claim 11, further comprising a display in electronic communication with the control system, the display showing the skin condition selected by the user.

16. The phototherapy device of claim 10, further comprising a display in electronic communication with the control system, the display showing a timer indicating a treatment time.

17. The phototherapy device of claim 1, further comprising a portable power source.

18. The phototherapy device of claim 1, wherein the skin condition is at least one of: acne, rosacea, wrinkles, inflammation, sun damage, bacteria, blemishes, or lesions.

19. The phototherapy device of claim 3, further comprising a detector configured to detect a fluorescence emission of a byproduct of bacteria on the skin exposed to ultraviolet light.

20. The phototherapy device of claim 19, wherein the fluorescence emission has a wavelength between 580 nanometers and 680 nanometers.

21. The phototherapy device of claim 19, wherein the detector is configured to generate a visual or audible signal when the fluorescence emission is detected.

22. The phototherapy device of claim 2, further comprising a detector configured to detect a fluorescence emission of a byproduct of bacteria on the skin exposed to ultraviolet light, wherein the control system is in electrical communication with the detector such that one or more operating parameters are affected by the amount of fluorescence emission detected.

23. The phototherapy device of claim 22, wherein the affected parameters are selected from the group consisting of duration and intensity.

24. The phototherapy device of claim 22, wherein at least one operating parameter is increased in response to detecting a relatively higher amount of fluorescence emission and decreased in response to detecting a relatively lower amount of fluorescence emission.

25. The phototherapy device of claim 3, wherein the second light source is configured to produce ultraviolet light having a wavelength between 360 nanometers and 365 nanometers.

26. The phototherapy device of claim 1, wherein the first light source comprises at least one laser.
